(19) United States
(12) Patent Application Publication
(10) Pub. No.: US 2012/0095566 A1
(43) Pub. Date: Apr. 19, 2012

(54) FLEXIBLE URETERAL STENT

(75) Inventor: James Teague, Spencer, IN (US)

(73) Assignee: BOSTON SCIENTIFIC SCIMED, INC., Maple Grove, MN (US)

(21) Appl. No.: 13/238,992

(22) Filed: Sep. 21, 2011

Related U.S. Application Data
(60) Provisional application No. 61/394,180, filed on Oct. 18, 2010.

(51) Int. Cl. A61F 2/04

(52) U.S. Cl. 623/23.7

(57) ABSTRACT

In one embodiment, a medical device includes an elongated member having a sidewall defining a lumen. The lumen extends through the member and defines a center line which extends through a center of the lumen in a direction longitudinal to the member. The sidewall defines a plurality of slots. Each successive slot is circumferentially offset about the sidewall. In another embodiment, a medical device includes a member defining a lumen extending through the member. Locations of an outer surface of the member are defined by a distance d from a proximal end of the member and an angle θ from a first point of a cross-section of the member. A sidewall of the member includes slots extending through the sidewall along planes extending from a line extending through a center of the lumen. Successive slots have their respective distances d successively farther from the proximal end of the member and their respective angles θ successively increasing.

### CROSS-REFERENCE TO RELATED APPLICATION

This application is a Nonprovisional of, and claims priority to, U.S. Patent Application No. 61/394,180, filed Oct. 18, 2010, entitled “FLEXIBLE URETERAL STENT”, which is incorporated by reference herein in its entirety.

### TECHNICAL FIELD

This disclosure relates generally to medical devices for draining fluids, and more specifically to stents including ureteral stents that are configured to extend from a kidney of a patient to a bladder of a patient.

### BACKGROUND

Medical devices are often used to drain fluids within a patient's body. For example, ureteral stents can be used to assist the drainage of fluids through the urinary system of a patient. Some known ureteral stents include a tubular member and are configured to assist the drainage of fluid from one part of the urinary system to another part of the urinary system. Some known ureteral stents are configured to extend from a patient's kidney to a patient's bladder. Such known ureteral stents assist to drain fluid through a urinary tract from the patient's kidney to the patient's bladder. The rigidity of some known ureteral stents may cause pain in the patient's urinary tract. For example, when a patient is walking or engaging in other movements involving the pelvis, the patient's urinary tract may move against the ureteral stent. The rigidity of the ureteral stent may not allow the ureteral stent to move with the patient's urinary tract, resulting in painful pressure in the patient's urinary tract as the ureteral stent maintains its shape within the urinary tract.

Some approaches have focused on stent designs that include spiral cutting of a tube wall all the way through the wall, creating a “telephone cord” effect. This method has shown difficulty in placing the stent inside the ureter due to stretching and/or coil jumping as the stent is passed through the tight ureteral orifice and up the ureter.

### SUMMARY

In one embodiment, a medical device includes an elongated member having a sidewall defining a lumen. The lumen extends through the member and defines a center line which extends through a center of the lumen in a direction longitudinal to the member. The sidewall defines a plurality of slots. Each successive slot is circumferentially offset about the sidewall. In another embodiment, a medical device includes a member defining a lumen extending through the member. Locations of an outer surface of the member are defined by a distance d from a proximal end of the member and an angle θ from a first point of a cross-section of the member. A sidewall of the member includes slots extending through the sidewall along planes extending from a line extending through a center of the lumen. Successive slots have their respective distances d successively farther from the proximal end of the member and their respective angles θ successively increasing.

The details of one or more implementations are set forth in the accompanying drawings and the description below. Other features will be apparent from the description and drawings, and from the claims.

### BRIEF DESCRIPTION OF THE DRAWINGS

FIG. 1 is a perspective view of a stent according to an embodiment of the invention.

FIG. 2 is another perspective view of the stent according to an embodiment of the invention.

FIG. 3A is a cross-sectional view of the elongated member according to an embodiment of the invention.

FIG. 3B is another cross-sectional view of the elongated member according to an embodiment of the invention.

FIG. 3C is another cross-sectional view of the elongated member according to an embodiment of the invention.

FIG. 4A is a side cutout view of the elongated member according to an embodiment of the invention.

FIG. 4B is another side cutout view of the elongated member according to an embodiment of the invention.

FIG. 5 is a flowchart of a method of manufacturing a stent according to an embodiment of the invention.

### DETAILED DESCRIPTION

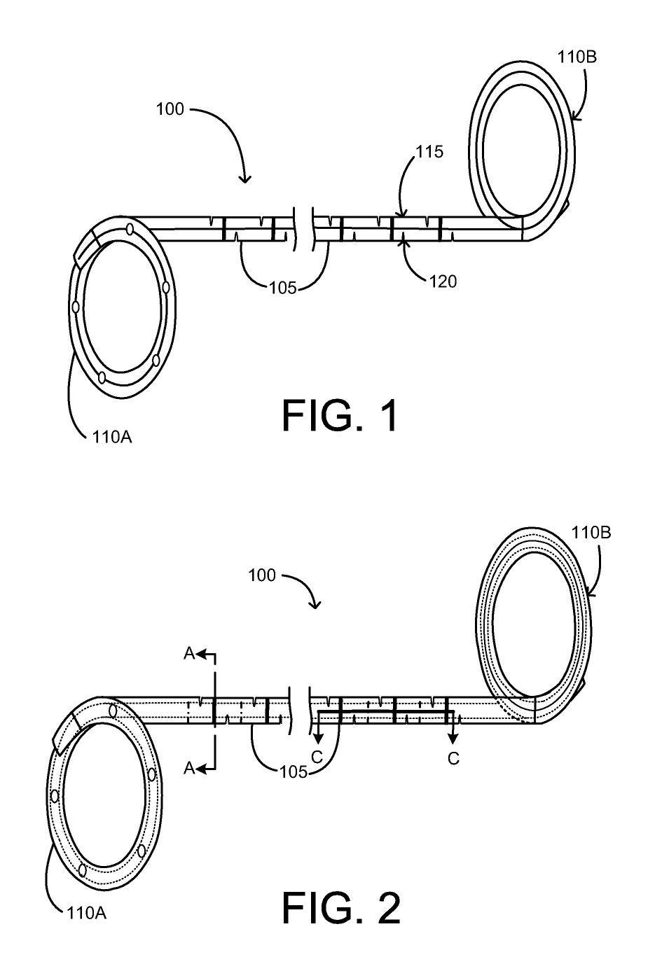

FIG. 1 is a perspective view of a medical device, such as a stent 100 according to an embodiment of the invention. The stent 100 includes an elongated member 105, which may be generally cylindrical. By “generally cylindrical,” the elongated member 105 is tubular, but need not be perfectly straight, and may bend at times. The elongated member 105, which will also includes slots, is discussed below. In some embodiments, the elongated member 105 is not generally cylindrical. For example, in some embodiments, the elongated member may have a square or rectangular cross-section.

The stent 100 may include one or more coiled portions 110A, 110B extending from opposite ends of the generally cylindrical elongated member 105. A proximal coiled portion 110A may be considered to extend from a proximal end of the generally elongated member 105, and a distal coiled portion 110B may be considered to extend from a distal end of the generally elongated member 105. The “proximal” end of the stent 100 may be considered the end which will enter a patient's kidney, and the “distal” end of the stent may be considered the end which will enter the patient's bladder.

In one embodiment, the stent 100 is configured to be placed within a body of the patient and to help facilitate the drainage of fluid from one portion of the body to another portion of the body.

In one embodiment, the stent 100 is a ureteral stent. In such an embodiment, the distal coiled portion 110B is configured to be disposed within a kidney of a patient and the proximal coiled portion 110A is configured to be disposed within a bladder of the patient. The generally cylindrical elongated member 105 is configured to extend within a ureter of the patient. In such an embodiment, the stent 100 is configured to help facilitate drainage of fluids such as urine within the urinary tract of the patient. Specifically, in such an embodiment, the stent 100 is configured to facilitate drainage from the kidney of the patient to the bladder of the patient.

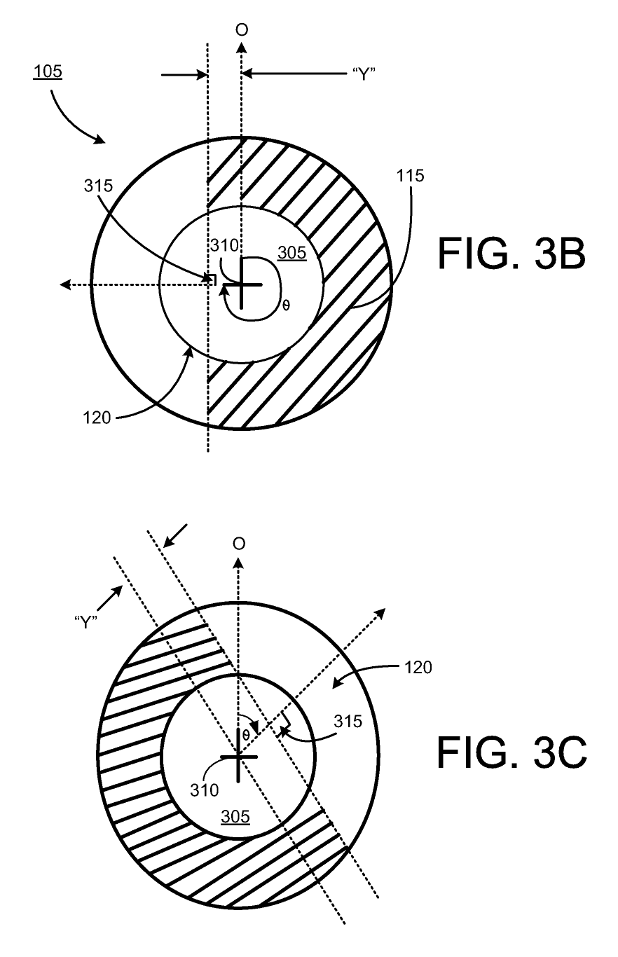

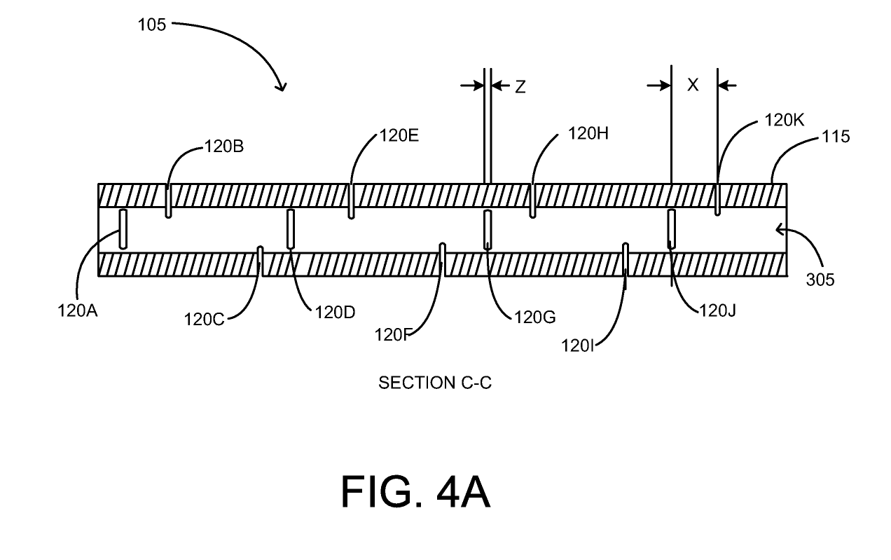

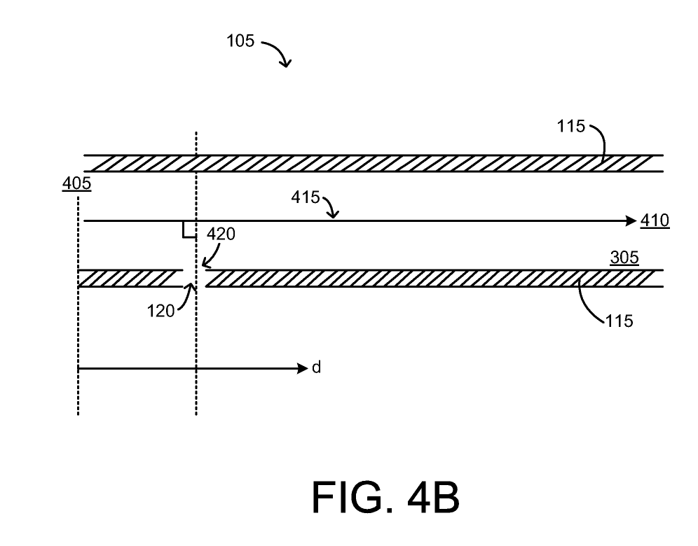

In some embodiments, the elongated member 105 of the stent 100 includes a wall or sidewall 115 which defines a lumen extending from the distal coiled portion 110B to the proximal coiled portion 110A. The lumen, which is shown in, and discussed with reference to, FIGS. 3A, 3B, 3C, 4A, and 4B, is configured to help facilitate the drainage of fluid within the body of the patient.

Additionally, the sidewall 115 includes slots 120 which are located along the length of the elongate member 105. The slots 120 extend from the lumen to outside the elongated member 105, and further facilitate the drainage of fluid within the patient's body. The slots 120 also provide flexibility to the stent 100, allowing the stent 100 to bend or flex in response to movement of the patient's ureter, reducing the pressure of the stent 100 within the patient's ureter and and/or trigon region of the patient's bladder, increasing the patient's comfort.

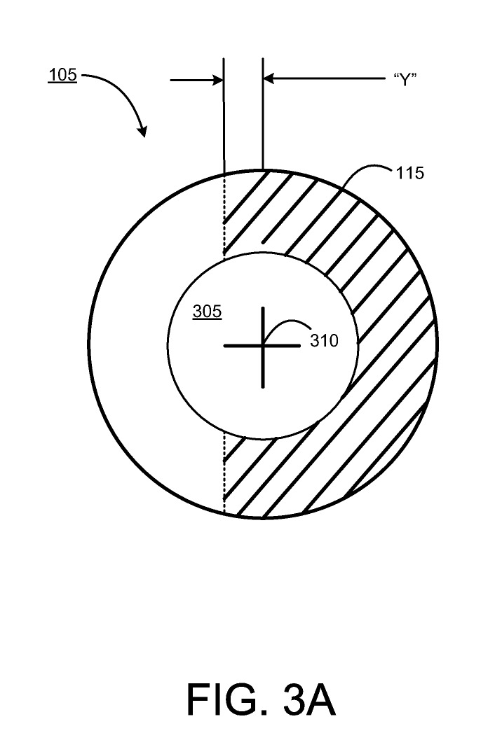

FIG. 2 is another perspective view of the stent 100 according to an embodiment of the invention. The arrows AA indicate the cross-sectional view shown in FIGS. 3A, 3B, and 3C, and the arrows CC indicate the cross-sectional view shown in FIGS. 4A and 4B.

FIG. 3A is a cross-sectional view of the elongated member 105 according to an embodiment of the invention. As shown in FIG. 3A, the sidewall 115 of the elongated member 105 defines a lumen 305. From the two-dimensional cross-sectional view of FIG. 3A, the sidewall 115 is generally circular, and the lumen 305 is generally circular; the lumen 305 extends along the longitude (or along a longitudinal axis) of the elongated member 105, and is generally cylindrical as defined by the sidewall 115. The lumen 305 may also extend into the coiled portions 110A, 110B shown in FIGS. 1 and 2. In other embodiments, the sidewall is not generally circular. Additionally, in other embodiments, the lumen defined by the sidewall is not generally circular. For example, the sidewall and the lumen defined by the sidewall may have a square, rectangular, or any other shape.

A center 310 of the lumen 305 may be defined by the inner surface of the sidewall 115. The center 310 may extend through a center of, and along the longitude of, the lumen 305 and the elongated member 105 to form a center line. The center line is shown in, and described further with reference to, FIG. 4B. The reference character Y indicates a distance from the center 310 from which the slots 120 are cut from the sidewall 115. The depth of the slots 120 from the outer surface of the sidewall 115 is (R−Y), where R is the radius of the elongated member 105, from the center 305 of the elongated member 105 to the outer surface of the sidewall 115. In some embodiments, Y has a value greater than zero, such as, for example, between 0.005 inches and 0.010 inches, so that the depth of the slots 120 is less than the radius of the elongated member 105. In such embodiments, more than half of the sidewall 115 remains opposite to each slot 120. In other embodiments, less than half of the sidewall 115 remains opposite to each slot 120. The geometry of the slots 120 with reference to the center 310 is described further with reference to FIGS. 3B and 3C.

FIG. 3B is another cross-sectional view of the elongated member 105 according to an embodiment of the invention. This cross-sectional view shows the slot 120 cut from the elongated member 105. The locations of the slots 120 rotate as the cross-section moves longitudinally along the elongated member 105. The locations of the slots may be defined with reference to an angle around the lumen 305 or sidewall 115. In this embodiment, an angle θ is defined from a first point, line, ray or angle, which may be defined as 0 (zero). In the two-dimensional cross-sectional perspective, the 0 line or angle may be a first line or ray extending from the center 310. In a three-dimensional perspective, the 0 line or angle may also be a line or ray, and may extend perpendicularly from the center line which extends longitudinally through the center 310 of the lumen 305 and elongated member 115.

The location or rotation of the slot 120 within the sidewall 115 may be defined by a distance d from a proximal (or distal) end of the elongated member (this distance d is illustrated in FIG. 4B), and by the angle θ with reference to the 0 line or angle. In the embodiment shown in FIG. 3B, the angle θ is two-hundred seventy (270) degrees. The slot 120 cuts into the sidewall 115 beginning at the outer surface of the sidewall 115 at the point indicated by the angle θ, and extends into the sidewall 115 of the elongated member 105 for a depth of (R−Y). In the illustrated embodiment, the slot 120 extends into the sidewall 115 of the elongated member 105 along a plane which is perpendicular to the center line and which includes the line or ray extending along the angle θ. The slot 120 ends at a line which is the distance Y from the center 310, and is at a right angle 315 to the line or ray extending along the angle θ. While the slot 120 in this embodiment extends into the sidewall 115 of the elongated member 105 perpendicularly to the center line and the longitude of the elongated member, the slot 120 may also extend into the sidewall 115 of the elongated member 105 at other, non-perpendicular angles.

FIG. 3C is another cross-sectional view of the elongated member 105 according to an embodiment of the invention. In this embodiment, the slot 120 shown in FIG. 3C may be a different distance d from the proximal end of the elongated member 105 than the slot 120 shown in FIG. 3B. Also in this embodiment, the slot 120 is circumferentially offset about the sidewall 105 from the slot 120 shown in FIG. 3B. In this embodiment, the angle θ is approximately forty-five (45) degrees. The slot 120 cuts into the sidewall 115 beginning at the outer surface of the sidewall 115 at the point indicated by θ, and extend into the sidewall of the elongated member 105 for a depth of (R−Y).

While FIGS. 3B and 3C show angles for the slots 115 of forty-five (45) and two-hundred seventy (270) degrees, the slots 120 may be any angle in the elongated member 105, and the progression of the angles θ between successive slots 120 may be any value, such as forty-five (45) degrees, between forty (40) and fifty (50) degrees, ninety (90) or less than ninety (90) degrees, or less than one-hundred eighty (180) degrees. Also, the progression of the angles θ may change across different intervals in the elongated member 105, allowing the elongated member 105 to bend more in one direction than another in portions of the elongated member 105, and/or the distance between successive slots 120 may change across different portions of the elongated member 105, giving the elongated member 105 different ratios of flexibility to rigidity along different portions of the elongated member 105.

FIG. 4A is a side cutout view of the elongated member 105 according to an embodiment of the invention. In this embodiment, the elongated member 105 is generally cylindrical. As shown in FIG. 4A, the sidewall 115 defines the lumen 305. The sidewall 115 defines a plurality of slots 120A, 120B, 120C, 120D, 120E, 120E, 120F, 120G, 120H, 120I, 120J, 120K. In this example, successive slots 120A, 120B, 120C, 120D, 120E, 120E, 120F, 120G, 120H, 120I, 120J, 120K are circumferentially offset or rotated by about ninety (90) degrees, with four slots 120A, 120B, 120C, 120D, 120E, 120E, 120F, 120G, 120H, 120I, 120J, 120K in each complete rotation about the elongated member (the slots 120 which would be closest to the viewer are not shown in FIG. 4A because of the cutout). In the embodiment shown in FIG. 1, as another example, successive slots 120A, 120B, 120C, 120D, 120E, 120E, 120F, 120G, 120H, 120I, 120J, 120K are circumferentially offset or rotated by about forty-five (45) degrees, with eight slots 120A, 120B, 120C, 120D, 120E, 120E, 120F, 120G, 120H, 120I, 120J, 120K in each complete rotation about the elongated member 105.

The spacing between each successive slot for the slots 120A, 120B, 120C, 120D, 120E, 120E, 120F, 120G, 120H, 120I, 120J, 120K is shown in FIG. 4A as X. The distance X may be measured between the centers of adjacent slots 120A, 120B, 120C, 120D, 120E, 120E, 120F, 120G, 120H, 120I, 120J, 120K, between the distal portions or edges of adjacent slots 120A, 120B, 120C, 120D, 120E, 120E, 120F, 120G, 120H, 120I, 120J, 120K, between the proximal portions or edges of adjacent slots 120A, 120B, 120C, 120D, 120E, 120E, 120F, 120G, 120H, 120I, 120J, 120K, or between the nearer edges or adjacent slots 120A, 120B, 120C, 120D, 120E, 120E, 120F, 120G, 120H, 120I, 120J, 120K. The spacing X may, for example, be between about 0.25 and 0.75 inches. While the successive slots 120A, 120B, 120C, 120D, 120E, 120E, 120F, 120G, 120H, 120I, 120J, 120K in the embodiment shown in FIG. 4A have approximately equal spacing X between them, in other embodiments, the slots 120 may be more closely spaced in some portions of the elongated member 105, making the elongated member 105 more flexible in the portions where the slots 120 are more closely spaced, and/or the slots may be spaced farther apart in some portions of the elongated member 105, making the elongated member 105 more rigid in the portions where the slots are spaced farther apart. The slots 120 may, for example, be spaced more closely together at a distal portion of the stent 100 and/or elongated member 105, making the stent 100 more flexible as the stent 100 enters the patient's bladder, with the slots 120 being spaced farther apart at a proximal end of the stent 100 and/or elongated member 105, making the stent 100 more rigid and/or stiffer at the proximal end, facilitating passage and retention of urine in and to the patient's kidney. The slots 120 may also continue into either or both of the coils 110A, 110B, making the coil(s) 110A, 110B flexible within the patient's bladder and/or kidney.

The width of the slots 120A, 120B, 120C, 120D, 120E, 120E, 120F, 120G, 120H, 120I, 120J, 120K is shown in FIG. 4A as Z, and may be measured between edges of the slots 120A, 120B, 120C, 120D, 120E, 120E, 120F, 120G, 120H, 120I, 120J, 120K. The width Z may be about the same for all the slots 120A, 120B, 120C, 120D, 120E, 120E, 120F, 120G, 120H, 120I, 120J, 120K, or the slots 120A, 120B, 120C, 120D, 120E, 120E, 120F, 120G, 120H, 120I, 120J, 120K may have different widths Z. Slots 120A, 120B, 120C, 120D, 120E, 120E, 120F, 120G, 120H, 120I, 120J, 120K with greater widths Z may provide more flexibility to the adjacent portions of the elongated member 105, whereas slots 120A, 120B, 120C, 120D, 120E, 120E, 120F, 120G, 120H, 120I, 120J, 120K with lesser widths Z may provide more rigidity and/or durability to the adjacent portions of the elongated member 105.

FIG. 4B is another side cutout view of the elongated member 105 according to an embodiment of the invention. As shown in FIG. 4B, the sidewall 115 of the elongated member 105 defines the lumen 305. Points or locations on the elongated member 105 and/or sidewall 115 may be defined and/or identified by a distance d, which may be measured from a proximal portion 405 of the elongated member 105 to a distal portion 410 of the elongated member 105. The lumen 305 may define a center line 410. The center line 410 extends through a center 310 (shown in FIGS. 3A, 3B, 3C) of the lumen 305 in a direction longitudinal to the elongated member 105. The center line 410 may be considered a longitudinal axis of the elongated member 105. The distance d may be measured along a line parallel to the center line 415, and may even be measured along the center line 415 itself.

The slots 120 may be formed along, and/or parallel to, a plane 420 which is perpendicular to the center line 410. The plane 420 extends from the center line 415, and/or is cross-sectional to the elongated member 105.

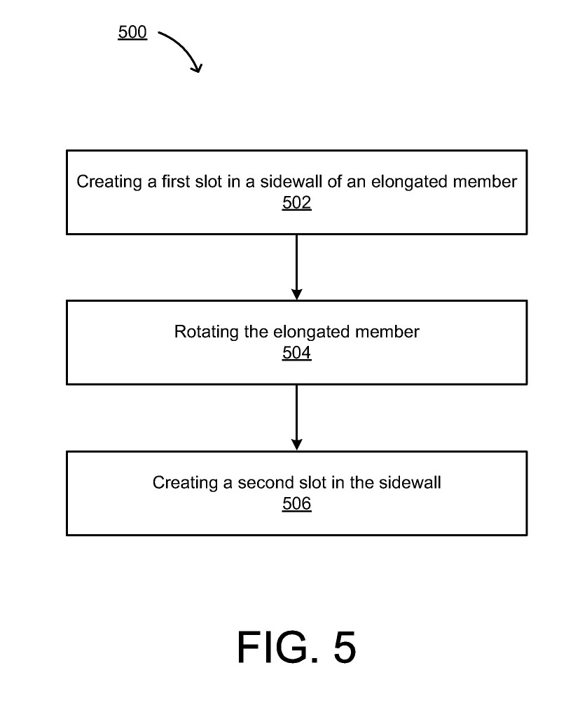

FIG. 5 is a flowchart of a method 500 of manufacturing a stent 100 according to an embodiment of the invention. In this embodiment, the method 500 includes creating a first slot 120 in a sidewall 115 of an elongated member 105 (step 502), rotating the elongated member 105 along a longitudinal axis (step 504), and creating a second slot 120 in the sidewall 115, the second slot 120 being distal to the first slot 120 along the longitudinal axis and being circumferentially offset about the sidewall 115 (step 506).

The slots 120 may be created in the elongated member 105 by, for example, cutting the slots 120 into the elongated member with a knife or laser. The slots 120 may be cut into the elongated member 105 with each rotation of the elongated member along the longitudinal axis, according to an embodiment. According to another embodiment, the stent 100 may be manufactured using a mold, such as an injection molding technique, with the mold including the slots 120, obviating the need for rotating the elongated member 105 along the longitudinal axis. The stent 100 may be monolithic or unitarily formed, or the different portions, such as the elongated member 105 and coils 110A, 110B, may be coupled together.

In an embodiment, the elongated member 105 may be generally cylindrical.

In an embodiment, the creating the first slot may include creating the first slot 120 in the sidewall 115 of the elongated member 105, the first slot 120 being perpendicular to a longitudinal axis of the elongated member 105, and the creating the second slot 120 may include creating the second slot 120 in the sidewall 115, the second slot 120 being perpendicular to the longitudinal axis, distal to the first slot 120 along the longitudinal axis, and circumferentially offset about the sidewall 115.

In an embodiment, the method 500 may further include coupling coiled portions 110A, 110B to each of a proximal end and a distal end of the elongated member 105. In another embodiment, the method 500 includes forming coiled portions 110A, 110B at the proximal and distal ends of the elongated member 105.

In some embodiments, the stent 100 is constructed of a biocompatible material. For example, in some embodiments, the stent 100 is constructed of a biocompatible plastic, such as, but not limited to, polyester, nylon-based biocompatible polymers, polytetrafluoroethylene polymers, silicone polymers, polyurethane polymers, polyethylene polymers, and thermoplastic polymers. In one embodiment, the stent 100 is constructed of ethylene vinyl acetate.

In one embodiment, a medical device includes an elongated member having a sidewall that defines a lumen. The lumen extends through the member and defines a center line which extends through a center of the lumen in a direction longitudinal to the member. The sidewall defines a plurality of slots. Each successive slot is circumferentially offset about the sidewall.

In one embodiment, the elongated member is generally cylindrical. In one embodiment, the slots extend through less than half of a cross-section of the member. IN one embodiment, the slots are located in planes which are perpendicular to the center line. In one embodiment, the medical device includes a coiled portion extending from one end of the generally cylindrical elongated member. In one embodiment, the medical device includes coiled portions extending from each end of the generally cylindrical elongated member.

In some embodiments, a medical device includes a member defining a lumen extending through the member. Locations of an outer surface of the member being defined by a distance d from a proximal end of the member and an angle θ from a first point of a cross-section of the member. A sidewall of the member includes slots extending through the sidewall along planes extending from a line extending through a center of the lumen. Successive slots have their respective distances d successively farther from the proximal end of the member and their respective angles θ successively increasing.

In one embodiment, the member is generally cylindrical. In one embodiment, the increase in the angle θ between successive slots is less than one-hundred eighty (180) degrees. In one embodiment, the increase in the angle θ between successive slots is approximately ninety (90) degrees. In one embodiment, the increase in the angle θ between successive slots is less than ninety (90) degrees. In one embodiment, the increase in the angle θ between successive slots between forty (40) and fifty (50) degrees.

In one embodiment, the slots extend through less than half of the cross-section of the member. In one embodiment, the planes along which the slots extend include cross-sectional planes which are perpendicular to the line extending through the center of the lumen. In one embodiment, the medical device includes a coiled portion extending from one end of the generally cylindrical elongated member. In one embodiment, the medical device includes coiled portions extending from each end of the generally cylindrical elongated member.

In one embodiment, a method of manufacturing a medical device includes (a) creating a first slot in a sidewall of an elongated member of the medical device; and (b) creating a second slot in the sidewall, the second slot being distal to the first slot along the longitudinal axis and being circumferentially offset about the sidewall. In one embodiment, the elongated member is generally cylindrical. In one embodiment, the creating the first slot includes creating the first slot in the sidewall of the elongated member, the first slot being perpendicular to a longitudinal axis of the elongated member; and the creating the second slot includes creating the second slot in the sidewall, the second slot being perpendicular to the longitudinal axis, distal to the first slot along the longitudinal axis, and circumferentially offset about the sidewall. In one embodiment, the method includes rotating the elongated member along a longitudinal axis to present a location on the sidewall for creating the second slot. In another embodiment, the method includes coupling coiled portions to each of a proximal end and a distal end of the elongated member.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A medical device comprising:
an elongated member having a sidewall defining a lumen, the lumen extending through the member and defining a center line which extends through a center of the lumen in a direction longitudinal to the member, the sidewall defining a plurality of slots, each successive slot being circumferentially offset about the sidewall.

2. The medical device of claim 1, wherein the elongated member is generally cylindrical.

3. The medical device of claim 1, wherein the slots extend through less than half of a cross-section of the member.

4. The medical device of claim 1, wherein the slots are located in planes which are perpendicular to the center line.

5. The medical device of claim 1, further comprising a coiled portion extending from one end of the generally cylindrical elongated member.

6. The medical device of claim 1, further comprising coiled portions extending from each end of the generally cylindrical elongated member.

7. A medical device comprising:
a member defining a lumen extending through the member, locations of an outer surface of the member being defined by a distance d from a proximal end of the member and an angle θ from a first point of a cross-section of the member;
wherein a sidewall of the member includes slots extending through the sidewall along planes extending from a line extending through a center of the lumen, and
successive slots have their respective distances d successively farther from the proximal end of the member and their respective angles θ successively increasing.

8. The medical device of claim 7, wherein the member is generally cylindrical.

9. The medical device of claim 7, wherein the increase in the angle θ between successive slots is less than one-hundred eighty (180) degrees.

10. The medical device of claim 7, wherein the increase in the angle θ between successive slots is approximately ninety (90) degrees.

11. The medical device of claim 7, wherein the increase in the angle θ between successive slots is less than ninety (90) degrees.

12. The medical device of claim 7, wherein the increase in the angle θ between successive slots between forty (40) and fifty (50) degrees.

13. The medical device of claim 7, wherein the slots extend through less than half of the cross-section of the member.

14. The medical device of claim 7, wherein the planes along which the slots extend include cross-sectional planes which are perpendicular to the line extending through the center of the lumen.

15. The medical device of claim 7, further comprising a coiled portion extending from one end of the generally cylindrical elongated member.

16. The medical device of claim 7, further comprising coiled portions extending from each end of the generally cylindrical elongated member.

17. A method of manufacturing a medical device comprising:
creating a first slot in a sidewall of an elongated member of the medical device;
and
creating a second slot in the sidewall, the second slot being distal to the first slot along the longitudinal axis and being circumferentially offset about the sidewall.

18. The method of claim 17, wherein the elongated member is generally cylindrical.

19. The method of claim 17, wherein:
the creating the first slot includes creating the first slot in the sidewall of the elongated member, the first slot being perpendicular to a longitudinal axis of the elongated member; and
the creating the second slot includes creating the second slot in the sidewall, the second slot being perpendicular to the longitudinal axis, distal to the first slot along the longitudinal axis, and circumferentially offset about the sidewall.

20. The method of claim 17, further comprising rotating the elongated member along a longitudinal axis to present a location on the sidewall for creating the second slot.
